(19) United States
(12) Patent Application Publication
(10) Pub. No.: US 2018/0140710 A1
(43) Pub. Date: May 24, 2018

(54) POLYMERS AND POLYMERIC ASSEMBLIES FOR PEPTIDE AND PROTEIN ENCAOSULATION AND RELEASE, AND METHODS THEREOF

(71) Applicant: University of Massachusetts, Boston, MA (US)

(72) Inventors: Sankaran Thayumanavan, Amherst, MA (US); Jeanne A Hardy, Amherst, MA (US)

(21) Appl. No.: 15/568,468

(22) PCT Filed: May 3, 2016
(86) PCT No.: PCT/US2016/030601

§ 371 Date: Oct. 21, 2017

Related U.S. Application Data
(60) Provisional application No. 62/156,646, filed on May 4, 2015.

(51) Int. Cl. A61K 47/58; A61K 47/69; A61K 9/51

(52) U.S. Cl. CPC A61K 47/58; G01R 33/46; A61K 9/51; A61K 47/6903

(57) ABSTRACT

The invention provides polymers and polymeric nanogels that stably encapsulate peptides and proteins, which are controllably released upon degradable of the nano-structures in response to specific microenvironment, and compositions and methods of preparation and use thereof.

### PRIORITY CLAIMS AND RELATED PATENT APPLICATIONS

This application claims the benefit of priority from U.S. Provisional Application Ser. No. 62/156,646, filed on May 4, 2015, the entire content of which is incorporated herein by reference in its entirety.

### STATEMENT OF FEDERALLY SPONSORED RESEARCH

The United States Government has certain rights to the invention pursuant to Grant No. CHE-1307118 awarded by the National Science Foundation and GRANT NO. CA169140 awarded by the National Institutes of Health.

### TECHNICAL FIELDS OF THE INVENTION

The invention generally relates to polymers and polymer-based nano-structures. More particularly, the invention relates to polymers and polymeric nanogels that stably encapsulate peptides and proteins, which are controllably released upon degradation of the nano-structures in response to specific microenvironment, and compositions and methods of preparation and use thereof.

### BACKGROUND OF THE INVENTION

Proteins perform vital biological functions, ranging from gene regulation to catalysis of metabolic reactions and cell signaling to programmed cell death. Proteins are widely used as therapeutics because they often exhibit higher specificity and offer more nuanced functions than can be achieved by small synthetic drugs. Unfortunately, many of the most biologically important proteins have inherent liabilities for manipulation and direct administration, including complications such as tremendous flexibility and a metastable “folded” state, large size, propensity to aggregate, susceptibility to oxidation or degradation, and triggering an immune resopnse. (Matsumoto, et al. 1998 Trends in Cell Biol. 8, 318-323; Darnell 1997 Science 277, 1630-1635; Ziegler 1988 Drug Metab. Revi. 19, 1-32; Lin, et al. 2001 Drug Metab. Dispos. 29, 368-374; Kim, et al. 2010 Biochim. Biophys. Acta 1802, 396-405; McKay, et al. 2007 Oncogene 26, 3113-3121; Hockenbery, et al. 1990 Nature 348, 334-336; Cardone, et al. 1998 Science 282, 1318-1321; Gu, et al. 2011 Chem. Soc. Rev. 40, 3638-3655; Leader, et al. 2008 Nat. Rev. Drug Discov. 7, 21-39; Rogers, et al. 2005 J. Am. Chem. Soc. 127, 10016-10017; Froidevaux, et al. 2002 Biopolymers 66, 161-183.)

Recent years have seen increasing interests in molecular systems that enable stable encapsulation of a guest molecule in one environment and then controlled release of the guest molecule in a different environment. The development of protein-polymer nanoconjugates to stabilize and deliver proteins is gaining tremendous interest. In particular, there is a great need for developing encapsulation systems for proteins as the guest molecules, because imbalance in protein activity is the primary reason for most human pathology. (Kerbel, et al. 2002 Nat. Rev. Cancer 2, 727-739; Harries, et al. 2002 Endocr.-Relat. Cancer 9, 75-85; Schrama, et al. 2006 Nat. Rev. Drug Discov. 5, 147-159; Gauthier, et al. 2010 Polym. Chem. 1, 1352-1373.)

Traditionally, conjugation of proteins to polymeric nanocarriers has been quite challenging due to the propensity of proteins to denature during polymer conjugation, which often requires using organic solvents and harsh synthesis conditions. (Murthy, et al. 2003 Proc. Natl. Acad. Sci. 100, 4995-5000, Cohen, et al. 2009 Bioconjugate Chem. 20, 111-119.)

During the past decade researchers have explored different approaches for conjugating proteins to telechelic, branched, star polymers and polymeric supramolecular molecules without compromising the activity or integrity of the protein. A commonly employed approach is the covalent conjugation of polymeric reactive side chains with surface exposed residues such as lysines and non-disulfide bonded cysteines. (Gonzalez-Toro, et al. 2013 Eur. Polym. J. 49, 2906-2918; Kalia, et al. 2010 Curr. Org. Chem. 14, 138-147; Grover, et al. 2010 Curr. Opin. Chem. Biol. 14, 818-828; Le Droumaguet, et al. 2010 Polym. Chem. 1, 563-598; Broyer, et al. 2011 Chem. Commun. 47, 2212-2226; Velonia 2010 Polym. Chem. 1, 944-952; Van Baal, et al. 2005 Angew. Chem. Int. Ed. 44, 5052-5057; Sletten, et al. 2009 Angew. Chem. Int. Ed. 48, 6974-6998; Matsumoto, et al. 2013 Polym. Chem. 4, 2464-2469.)

Since proteins may contain more than one reactive residue exposed to the solvent or have poor accessibility, there is little control over the conjugation site. To circumvent this, researchers have developed genetically modified proteins containing a single cysteine or lysine residue available for conjugation, but such modifications may have significant implications on the stability or function of the protein. Inverse microemulsion methods are also commonly employed where the protein, monomer, and crosslinker in the aqueous phase are dispersed in the organic phase and polymerization is carried out around the protein. (Murthy, et al. 2003 Proc. Natl. Acad. Sci. 100, 4995-5000; Cohen, et al. 2009 Bioconjugate Chem. 20, 111-119. achelder, et al. 2008 J. Am. Chem. Soc. 130, 10494-10495; Beaudette, et al. 2009 J. Am. Chem. Soc. 131, 10360-10361; Bachelder, et al. 2008 Mol. Pharmaceutics 5, 876-884.)

Meanwhile, controlled radical polymerization (CRP) methods are also gaining popularity and emerging as a powerful tool among researchers where proteins are modified with a moiety capable of chain initiation. Despite this, exposure to organic/aqueous phases and separation of unreacted protein or monomer can still be challenging, compromising protein integrity. Self assembly between polymeric nanoparticles and proteins is utilized as an alternative method to prevent irreversible chemical modification of proteins. Depending on surface charge, proteins can also be physically adsorbed by electrostatic forces onto the surface of polymeric vehicles. (Yan, et al. 2010 Nat. Nanotechnol. 5, 48-53; Wallat, et al. 2014 Polym. Chem. 4, 1545-1558; Bontempo, et al. 2005 J. Am. Chem. Soc. 127, 6508-6509; De. et al. 2008 J. Am. Chem. Soc. 130, 11288-11289; Ayame, et al. 2008 Bioconjugate Chem. 19, 882-890; Lee, et al. 2008 Biomaterials 29, 1224-1232; Cha, et al. 2005 Proteomics 5, 416-419; Gonzalez-Toro, et al. 2012 J. Am. Chem. Soc. 134, 6964-6967.)

Another challenge in this area is protecting proteins during delivery to prevent modification or inactivation. This is particularly applicable in biological milieu, such as blood, where proteins can be degraded by proteases, oxidized or subjected to a number of other insults. The goal of this work was to develop polymeric nanogels that are capable of protecting and delivering large, highly mobile, aggregation prone and enzymatically challenging proteins intracellularly. Caspases are one such class of targets. Casapses are also known to be an inherently sensitive in terms of their structural stability in non-native environments. Therefore, demonstration of encapsulating this protein and then activating it in its native environment would suggests applicability to a broad range of protein and peptide cargos.

Caspases are cysteine proteases that are known for their exquisite specificity for cleaving after particular aspartic acid residues and rapidly inducing apoptotic cell death. Their ability to trigger apoptosis makes them promising for selective cell killing, especially in cancer therapeutics. Among the family of apoptotic caspases, caspase-3 is of particular interest due to its major role in cleaving substrates during apoptosis as well as its high catalytic rate. Caspase-3 also presents five surface-exposed cysteine residues in each monomer (FIG. 9) which can be utilized as handles for attachment to polymeric materials using thiol chemistry. (Budihardjo, et al. 1999 Annu. Rev. Cell Dev. Biol. 15, 269-290; Shi 2002 Molecular Cell 9, 459-470; Chang, et al. 2000 Mol. Biol. Rev. 64, 821-846; Huang, et al. 2002 Mol. Pharmacol. 3, 569-577; McStay, et al. 2008 Cell Death Differ. 15, 322-331.)

Conjugation of caspases to polymeric materials is particularly attractive since it harnesses a cell's own biological processes to induce cell death. However, transport of active caspases can be dangerous as release in an erroneous location can lead to irreversible proteolytic damage or even unwanted apoptotic death. The therapeutic potential of caspases for inducing cell death could be harnessed if it were possible to silence caspase activity through polymer conjugation and then recover its activity upon response to a precise trigger.

Self cross-linked polymeric nanogels were recently reported to have the capability of these vehicles to stably encapsulate hydrophobic molecules and release them in response to a specific stimulus. However, no such strategy exists for hydrophilic molecules such as proteins. (Ryu, et al. 2010 J. Am. Chem. Soc. 132, 17227-17235; Jiwpanich, et al. 2010 J. Am. Chem. Soc. 132, 10683; Ryu, et al. 2010 J. Am. Chem. Soc. 132, 8246-8247.)

It was desired to develop a self-crosslinking based strategy to encapsulate proteins using polymers, where the redox responsive characteristics of the polymer are utilized to first silence and later activate the encapsulated proteins. An urgent unmet need remains in developing polymeric nanogels that encapsulate a guest molecule stably in one environment and then release it in a different environment in controlled fashion.

### SUMMARY OF THE INVENTION

The invention provides a novel crosslinked polymeric nanogel delivery system comprised of a nanogel-protein conjugate, which can stably transport a protein across a cell membrane and then intracellularly release the protein with its biological activity intact. The invention also provides simple and reliable synthetic techniques for making the polymers, polymeric nanogels and protein delivery vehicles disclosed herein.

The invention provides a methodology to “wrap-up” the structure of a protein by polymer conjugation, simultaneously protecting its delicate folded state and silencing its enzymatic activity. For example, the study disclosed herein demonstrated that thiol-disulfide exchange reactions are capable of silencing the enzymatic activity of a caspase, protecting the protein from denaturation during synthesis and delivery, and finally enabling intracellular delivery of an active caspase with the nanogel vehicles.

The method allows recovery of activity only upon cytosolic uptake, which triggers the release from the polymer. For example, redox-responsive polymeric nanogels were developed based on the collapse and self cross-linking of a limited number of pyridyldisulfide (PDS) side chains groups. Caspase-3, an apoptosis-inducing protein, was successfully conjugated on either the interior or the surface of nanogels by a thiol-disulfide exchange reaction between unreacted polymer PDS groups and surface exposed cysteine residues in caspase-3. As demonstrated herein, this approach allowed reversible release and recovery of upto 80% to 90% of caspase activity. Evidence showed that nanogel-caspase conjugates were observed within cells after 4 hours of treatment and cell death was induced in 70-80% of cells shortly thereafter.

In one aspect, the invention generally relates to a crosslinked polymeric nanogel-protein conjugate adapted to stably transporting a protein across a cell membrane and then intracellularly releasing the protein with intact biological activity.

In another aspect, the invention generally relates to a method for controlled delivery of a protein to a target biological site inside a cell. The method includes: providing a crosslinked polymeric nanogel-protein conjugate adapted to stably transporting a protein across the cell membrane and then intracellularly releasing the protein with intact biological activity; delivering the crosslinked polymeric nanogel-protein conjugate intracellularly to the target biological site; and causing a dissociation of the protein from the polymeric nanogel-protein conjugate resulting in intracellular release of the protein at the target biological site.

In certain preferred embodiments, the protein is securely encapsulated inside a nanoaggregate formed by the self-crosslinked polymeric nanogel. In certain embodiments, the protein is encapsulated inside the nanoaggregate by one or more disulfide linkages.

In certain preferred embodiments, the protein is securely conjugated on a surface of a nanoaggregate formed by the self-crosslinked polymeric nanogel. In certain preferred embodiments, the protein is conjugated to the surface of the nanoaggregate by one or more disulfide linkages.

Any suitable proteins may be transported and released intracellularly according to the method disclosed herein. Exemplary proteins include caspase proteins (e.g., Caspase-3), RNAse-H, azoreductase and gultamine synthetase.

The crosslinked polymeric nanogel-protein conjugate may be further functionalized with a cell penetrating peptide, e.g., RRR (see FIG. 1), trans-activating transcriptional activator (Tat) peptide, folic acid, Arg-Gly-Asp (RGD), methotrexate or target-specific antibodies).

The crosslinked polymeric nanogel-protein conjugate may be further functionalized with a targeting ligand such as an antibody protein, a peptide, or a small molecule.

In certain preferred embodiments, the crosslinked polymeric nanogel-protein conjugate is functionalized with a peptide as a targeting ligand.

Any suitable polymers may be utilized to form the polymeric nanogel, for example, a random copolymer. In certain embodiments, the polymeric nanogel is formed by reversible addition-fragmentation chain transfer polymerization of oligo(ethylene glycol) methacrylate and pyridyldisulfide methacrylate.

### BRIEF DESCRIPTION OF THE DRAWINGS

FIG. 1. Covalent conjugation of caspase-3 in the interior or on the surface of polymeric redox sensitive nanogels

FIG. 2. (A) DLS of nanogel-caspase conjugates (B) DLS of nanogel-caspase^{RRR} conjugates (C) ζ-potential of nanogel-caspase conjugates (D) ζ-potential of nanogel-caspase^{RRR} conjugates.

FIG. 3. SDS-PAGE gel validating the nanogel-caspase conjugation through reducible disulfide linkages. (A) Nanogel-caspase conjugates under non-reducing conditions. (B) Nanogel-caspase conjugates under reducing conditions. (C) Nanogel-caspase^{RRR}conjugates under non-reducing conditions. (D) Nanogel-caspase^{RRR} under reducing conditions.

FIG. 4. Mass spectra of (A) caspase-3 (B) NG-Empty (C) NG-Caps-In (D) NG-Caps-Out.

FIG. 5. (A) Enzymatic activity of caspase-3 (B) caspase-3 percentage activity recovered from nanogel-caspase conjugates was measured under essentially non-reducing (0.5 mM DTT) or fully-reducing (100 mM DTT) conditions. At 0.5 mM DTT no disassembly of nanogels is observed, whereas at 100 mM DTT full disassembly is observed (C) enzymatic activity of caspase-3 (D) percent activity recovered from nanogel-caspase ^{RRR} conjugates. This experiment was performed in duplicate on each of two days.

FIG. 6. SDS-PAGE gel validating the nanogel-caspase conjugation through reducible disulfide linkages (A) Nanogel-caspase^{PEG} conjugates under non-reducing conditions (B) nanogel-caspase^{PEG} conjugates under reducing conditions (C) percent activity recovered from nanogel-caspase^{PEG} conjugates.

FIG. 7. Cellular internalization of the (A) NG-FITC-Casp-In (B) NG-FITC-Casp-Out (C) NG-FITC-Casp-In^{RRR} (D) NG-FITC-Casp-Out^{RRR} at 0.5 mg/mL on HeLa cells. Within each image set, top left is the FITC channel, which show green color for caspase-3 and top right is the DRAQ5 channel, which shows red color for the nucleus. Bottom left is the DIC image and bottom right is the overlap of all three. This experiment was performed with triplicate visualization on one day. One representative field is shown for each condition.

FIG. 8. Cell viability after 24 hours exposure of HeLa cells with the conjugates (A) nanogel-caspase conjugates (B) nanogel-caspase^{RRR}. * The concentration in the caspase-3 samples is the feed amount of caspase-3 used when preparing 0.1 mg/mL, 0.5 mg/mL and 1 mg/mL solutions. Nanogel:Caspase-3 (50:1). The experiment of FIG. 8A was performed in triplicate on one day. The experiment in FIG. 8B was performed in triplicate on two separate days. Data from one day is shown. The second day is FIG. 15.

FIG. 9. Surface exposed cysteine residues in caspase-3.

FIG. 10. NMR spectra of p(PEGMA-co-PDSMA).

FIG. 11. NMR spectrum of the CRRR peptide.

FIG. 12. Absorption spectra of nanogel-caspase conjugates synthesis. A) NG Empty (0.1 mg/mL) B) NG-FITC-Casp-In (0.1 mg/mL) C) NG-FITC-Casp-Out (0.1 mg/mL) D) NG Empty^{RRR} (0.05 mg/mL) E) NG-FITC-Casp-In^{RRR} (0.05 mg/mL) F) NG-FITC-Casp-Out^{RRR} (0.05 mg/mL).

FIG. 13. Caspase-3 activity recovered after an early protocol for the construction of caspase-containing nanogels involving lyophilization. 50 nM caspase-3 control and 50 nM released from nanogels.

FIG. 14. No cellular internalization after incubating the cells for 4 hours with FITC-caspase-3. Within each image set, top left is the FITC channel, which show green color for caspase-3 and top right is the DRAQ5 channel, which shows red color for the nucleus. Bottom left is the DIC image and bottom right is the overlap of all three.

FIG. 15. Apoptosis experiment performed with nanogel-caspase^{RRR} on day two.

FIG. 16. HeLa cells after incubation for 24 hours with nanogel-caspase conjugates. A) Untreated cells B) 1 mg/mL NG Empty C) 1 mg/mL NG-Casp-In D) 1 mg/mL NG-Casp-Out E) 1 ΞM staurosporine.

### DEFINITIONS

Definitions of specific functional groups and chemical terms are described in more detail below. General principles of organic chemistry, as well as specific functional moieties and reactivity, are described in “Organic Chemistry”, Thomas Sorrell, University Science Books, Sausalito: 2006. It will be appreciated that the compounds, as described herein, may be substituted with any number of substituents or functional moieties.

As used herein, “Cₓ-C_{y}” refers in general to groups that have from x to y (inclusive) carbon atoms. Therefore, for example, C₁-C₆ refers to groups that have 1, 2, 3, 4, 5, or 6 carbon atoms, which encompass C₁-C₂, C₁-C₃, C₁-C₄, C₁-C₅, C₂-C₃, C₂-C₄, C₂-C₅, C₂-C₆, and all like combinations. “C₁-C₁₅”, “C₁-C₂₀” and the likes similarly encompass the various combinations between 1 and 20 (inclusive) carbon atoms, such as C₁-C₆, C₁-C₁₂, C₃-C₁₂ and C₆-C₁₂.

As used herein, the term “alkyl”, refers to a hydrocarbyl group, which is a saturated hydrocarbon radical having the number of carbon atoms designated and includes straight, branched chain, cyclic and polycyclic groups. The term “hydrocarbyl” refers to any moiety comprising only hydrogen and carbon atoms. Hydrocarbyl groups include saturated (e.g., alkyl groups), unsaturated groups (e.g., alkenes and alkynes), aromatic groups (e.g., phenyl and naphthyl) and mixtures thereof.

As used herein, the term “Cₓ-C_{y} alkyl” refers to a saturated linear or branched free radical consisting essentially of x to y carbon atoms, wherein x is an integer from 1 to about 10 and y is an integer from about 2 to about 20. Exemplary Cₓ-C_{y} alkyl groups include “C₁-C₂₀ alkyl,” which refers to a saturated linear or branched free radical consisting essentially of 1 to 20 carbon atoms and a corresponding number of hydrogen atoms. Exemplary C₁-C₂₀ alkyl groups include methyl, ethyl, n-propyl, isopropyl, n-butyl, isobutyl, dodecanyl, etc.

As used herein, the term, “Cₓ-C_{y} alkoxy” refers to a straight or branched chain alkyl group consisting essentially of from x to y carbon atoms that is attached to the main structure via an oxygen atom, wherein x is an integer from 1 to about 10 and y is an integer from about 2 to about 20. For example, “C₁-C₂₀ alkoxy” refers to a straight or branched chain alkyl group having 1-20 carbon atoms that is attached to the main structure via an oxygen atom, thus having the general formula alkyl-O—, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

As used herein, the term “halogen” refers to fluorine (F), chlorine (CO, bromine (Br), or iodine (I).

### DETAILED DESCRIPTION OF THE INVENTION

The invention is based in part on the unexpected discovery of a novel crosslinked polymeric nanogel delivery system comprising nanogel-protein conjugate that is able to stably transport a protein across a cell membrane and then intracellularly release the protein with intact biological activity.

The polymers, polymeric nanogels and protein delivery vehicles of the invention can be prepared via simple and reliable synthetic techniques. The study disclosed herein demonstrated that thiol-disulfide exchange reactions are capable of silencing the enzymatic activity of a caspase, protecting the protein from denaturation during synthesis and delivery, and finally enabling intracellular delivery of an active caspase with the nanogel vehicles.

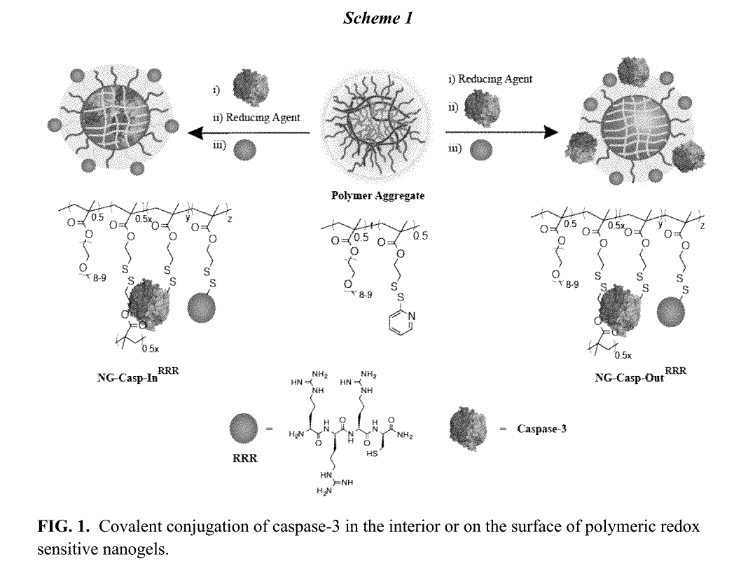

Exemplary polymeric nanogels were prepared to which caspase-3 was reproducibly conjugated either in the interior or on the nanogel surface through disulfide linkages (FIG. 1, Scheme 1). In addition, these nanogel-caspase conjugates were further functionalized with a cell penetrating peptide (a ligand to facilitate cellular uptake) and the enzymatic activity of the protein was evaluated upon dissociation from the nanogels.

The present disclosure demonstrates robust strategies to conjugate active enzymes to polymeric nanogels with responsive characteristics, where caspase-3 has been used as the active protein cargo. It was shown that: (i) the proteins can be attached to the surface of the nanogels or can be encapsulated within the polymeric nanogel. (ii) The activity of the protein is completely turned off in both these approaches, a feature that is useful when delivering cargos that could have deleterious consequences in off-target locations. (iii) The redox sensitive unlocking event causes the protein to be reactivated, allowing recovery of about 80% to about 90% of the activity. In addition to showing the versatility of these approaches, the fact that these have been demonstrated with a protein that is very prone to irreversible unfolding suggests that this approach is broadly applicable to other proteins. (iv) The recovery of activity under reducing conditions can be capitalized inside cells, where the reducing environment of the cytosol due to high glutathione concentrations is targeted as the triggering mechanism. This was discerned by the fact that both conjugation approaches provide robust cellular entry, protein release, and apoptotic activity. (v) The nanogel-protein conjugate was able to gain cellular entry, while the protein by itself did not enter the cells. These results, combined with the fact that the nanogel by itself is not cytotoxic, suggest that these conjugates are promising protein carriers. (vi) The protein conjugated to the surface of the nanogel is prone to proteolysis, while the encapsulated protein is not accessible to proteolytic enzymes.

The disclosure indicates that while both approaches are versatile for in vitro protein delivery, the encapsulation approach is more promising for future in vivo applications. It was further shown that: (vii) The nanogel-protein conjugates, decorated with cell-penetrating peptides, gain cellular entry much more rapidly compared to the unfunctionalized nanogels. However, the overall apoptotic efficiency of the unfunctionalized nanogels is comparable (or even better than) to those functionalized with RRR. These results show that the unfunctionalized nanogels end up in the cytosol more effectively, as these are not complicated by electrostatic association with cellular membranes. The fact that the unfunctionalized nanogels are slow in cellular uptake, but are more effective in releasing the cargo, bodes well for utilizing these vehicles for targeted delivery of other protein cargos. The disclosed approach is widely generalizable and applicable to the intracellular delivery of a wide range of therapeutic proteins for treatment of cancer, diabetes and genetic diseases.

In one aspect, the invention generally relates to a crosslinked polymeric nanogel-protein conjugate adapted to stably transporting a protein across a cell membrane and then intracellularly releasing the protein with intact biological activity.

In another aspect, the invention generally relates to a method for controlled delivery of a protein to a target biological site inside a cell. The method includes: providing a crosslinked polymeric nanogel-protein conjugate adapted to stably transporting a protein across the cell membrane and then intracellularly releasing the protein with intact biological activity; delivering the crosslinked polymeric nanogel-protein conjugate intracellularly to the target biological site; and causing a dissociation of the protein from the polymeric nanogel-protein conjugate resulting in intracellular release of the protein at the target biological site.

In certain preferred embodiments, the protein is securely encapsulated inside a nanoaggregate formed by the self-crosslinked polymeric nanogel. In certain embodiments, the protein is encapsulated inside the nanoaggregate by one or more disulfide linkages.

In certain preferred embodiments, the protein is securely conjugated on a surface of a nanoaggregate formed by the self-crosslinked polymeric nanogel. In certain preferred embodiments, the protein is conjugated to the surface of the nanoaggregate by one or more disulfide linkages.

Any suitable proteins may be transported and released intracellularly according to the method disclosed herein. Exemplary proteins include caspase proteins (e.g., Caspase-3), RNAse-H, azoreductase and gultamine synthetase.

The crosslinked polymeric nanogel-protein conjugate may be further functionalized with a cell penetrating peptide (e.g., RRR, Tat peptide, folic acid, RGD, methotrexate or target-specific antibodies).

The crosslinked polymeric nanogel-protein conjugate may be further functionalized with a targeting ligand such as an antibody protein, a peptide, or a small molecule.

In certain preferred embodiments, the crosslinked polymeric nanogel-protein conjugate is functionalized with a peptide as a targeting ligand.

Any suitable polymers may be utilized to form the polymeric nanogel, for example, a random copolymer. In certain embodiments, the polymeric nanogel is formed by reversible addition-fragmentation chain transfer polymerization of oligo(ethylene glycol) methacrylate and pyridyldisulfide methacrylate.

In certain embodiments, the crosslinked polymeric nanogel-protein conjugate comprising a block or random co-polymer comprising structural units of:

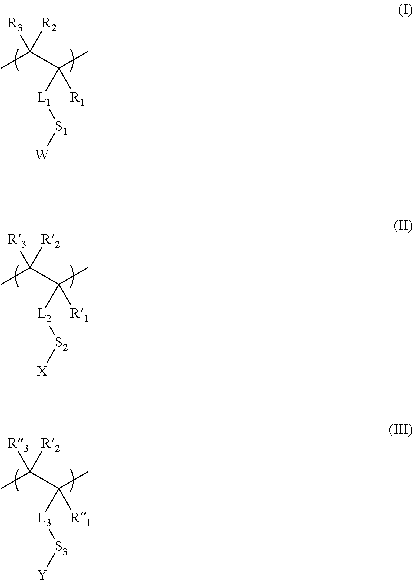

wherein

each of R₁, R′₁ and R″₁ is independently a hydrogen, C₁-C₁₂ alkyl group, or halogen;

each of R₂, R′₂, R″₂, R₃, R′₃ and R″₃ is independently a hydrogen, (C₁-C₁₆) alkyl, (C₁-C₁₆) alkyloxy, or halogen;

each of L₁, L₂ and L₃ is independently a linking group;

each of S₁, S₂ and S₃ is independently a single bond or a spacer group;

W comprises a hydrophilic group;

X comprises a crosslinking group; and

Y comprises a peptide or protein.

In certain preferred embodiemnts, the block or random co-polymer further comprises a structural unit of:

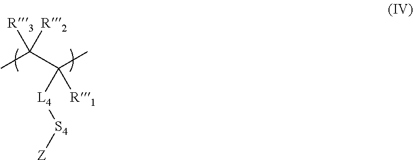

wherein

R″′₁ is a hydrogen, C₁-C₁₂ alkyl group, or halogen;

each of R″′₂ and R″′₃ is independently a hydrogen, (C₁-C₁₆) alkyl, (C₁-C₁₆) alkyloxy, or halogen;

L₄ is a linking group;

S₄ is a single bond or a spacer group; and

Z comprises a cell-penetrating group.

In certain preferred embodiemnts, the crosslinked polymeric nanogel-protein conjugate is covalently linked to a targeting group.

In certain embodiments, each of R₂, R′₂, R″₂, R₃, R′₃ and R″₃ is a hydrogen, and each of R₁, R′₁ and R″₁ is a methyl group.

In certain embodiments, each of L₁, L₂ and L₃ is

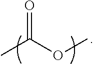

In certain embodiments, W comprises

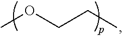

wherein p is an integer from about 1 to about 40 (e.g., from about 1 to about 36, from about 1 to about 24, from about 1 to about 12, from about 1 to about 6, from about 3 to about 36, from about 3 to about 24, from about 3 to about 12, from about 6 to about 36, from about 6 to about 24, from about 6 to about 12). W may include a charged group, a zwitterionic group.

Y may comprise any suitable peptide or protein to be delivered, for example,caspases, RNAse, insulin, or any other therapeutic protein.

In certain preferred embodiments, X comprises a disulfide moiety.

### EXAMPLES

### Preparation and Characterization of Nanogel-Caspase Conjugates

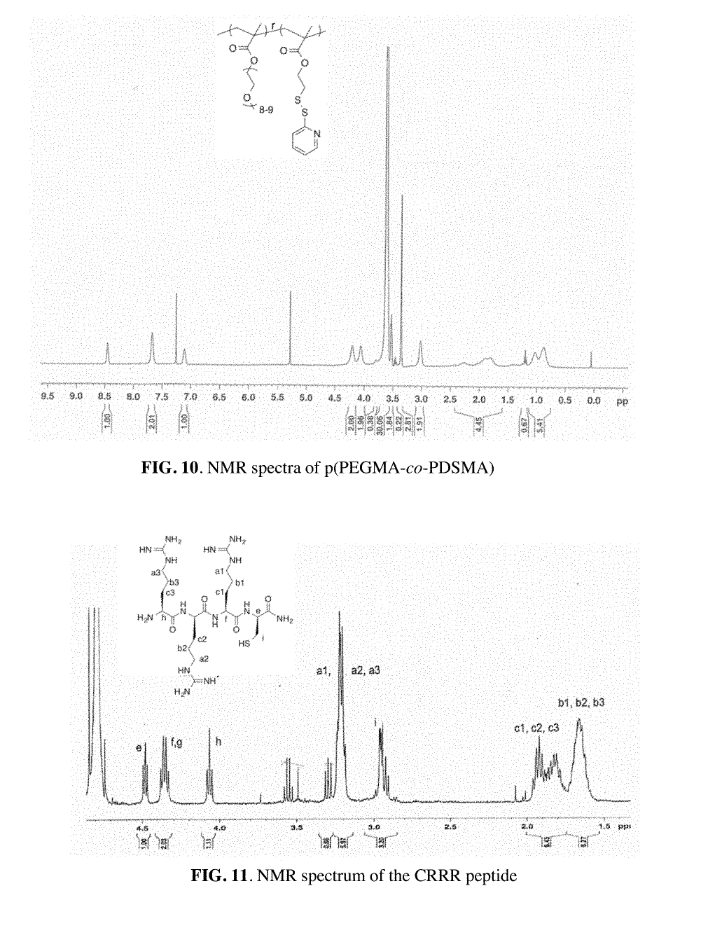

In order to form nanogels appropriate for conjugation and transport of hydrophilic cargos, it was essential to carefully design the polymer. The random copolymer nanogel precursor was obtained by the reversible addition-fragmentation chain transfer (RAFT) polymerization of oligo(ethylene glycol) (OEG) methacrylate and pyridyldisulfide (PDS) methacrylate. The feed ratio of the monomer was 50:50 and experimentally the resulting copolymer was found to contain 48% of the OEG units and 52% of the PDS groups as discerned by NMR (FIG. 10). In aqueous solution, this amphiphilic polymer forms nanoaggregates; these aggregates were locked using a self-crosslinking process.

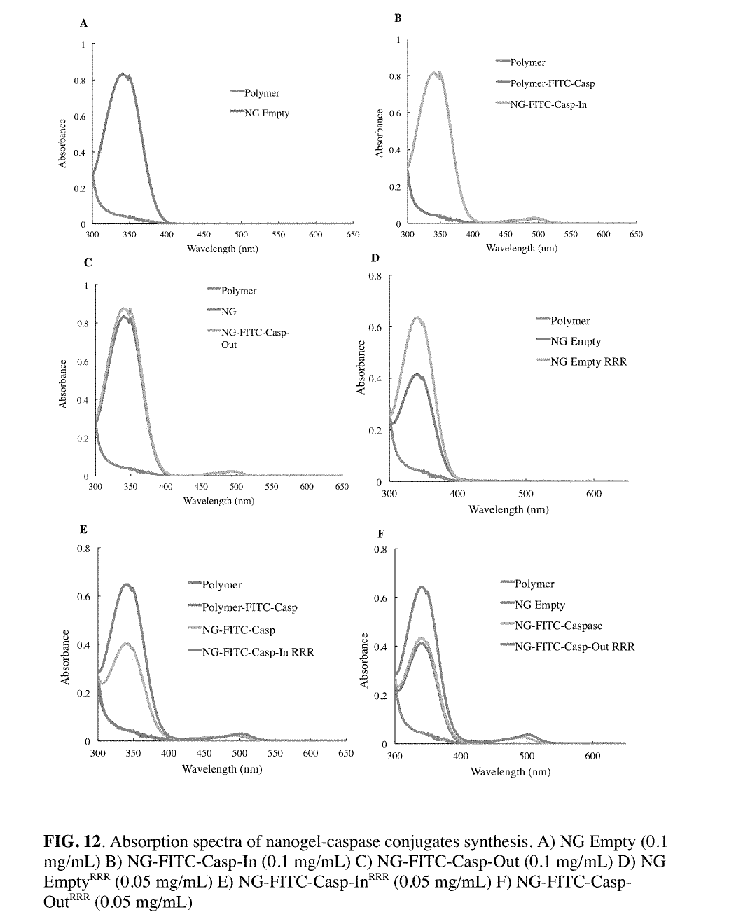

This self-crosslinking process was enabled by intra- and inter-chain disulfide cross-linking of the PDS groups in the presence of the reducing agent dithiothreitol (DTT). The nanogel formation process was monitored by tracing the absorption spectra of pyridothione (byproduct of the disulfide crosslinking) at 343 nm (FIG. 12). Based on the pyridothione released, the crosslink density of these nanogels was 18% (FIG. 12A). These nanogels were labeled as ‘NG-empty’, since these do not have any cargo molecules encapsulated inside.

The possibility of localizing and attaching the protein to the nanogel was investigated: either encapsulated within the crosslinked nanogel structure or decorated on the nanogel surface. To encapsulate caspase-3 within the interior, caspase-3 and the amphiphilic polymer were added simultaneously to aqueous media. As a result, caspase-3 was sequestered in the core of the aggregates during the self-assembly process of the polymer. The protein was then “wrapped-in” the assembly by initiating thiol-disulfide exchange reactions between PDS groups with addition of DTT, trapping caspase-3 within the core of these nanogels (FIG. 1, Scheme 1, NG-Casp-In).

It is likely that the exposed cysteines on the caspase also played a role in the efficient encapsulation of the proteins within the nanogel. To take advantage of the surface conjugation capabilities of these polymeric materials, first prepared was the cross-linked nanogels (NG-empty) and caspase-3 was then covalently attached on its surface using the cysteine residues of the protein and unreacted PDS groups (Scheme 1, NG-Casp-Out). This effectively decorated the nanogel with caspase-3 attached on the outside.

Due to their hydrophobic nature, it was expected that unreacted PDS groups might collapse into the core of the nanogels, but found that surface functionalization of these nanogels could still be achieved with reasonable efficiency. Also prepared were nanogels with cell penetrating capabilities by incorporating a cysteine-containing tri-arginine peptide on the surface of these nanogels to generate NG-Empty^{RRR}, NG-Casp-In^{RRR}, NG-Casp-Out^{RRR} (Scheme 1). (Rothbard, et al. 2004 1 Am. Chem. Soc. 126, 9506-9507; Nakase, et al. 2008 Adv. Drug Delivery Rev. 60, 598-607.)

Incorporation of the peptide on the surface was confirmed by further increase in the pyridothione absorption spectra (FIGS. 12D, E and F). Based on the pyridothione released, it was found that the presence or absence of caspase had no overall impact in the ability to conjugate a second molecular entity. Also, the crosslinking densities of these nanogels NG-Empty^{RRR}, NG-Casp-In^{RRR}, NG-Casp-Out^{RRR} were each found to be 18%.

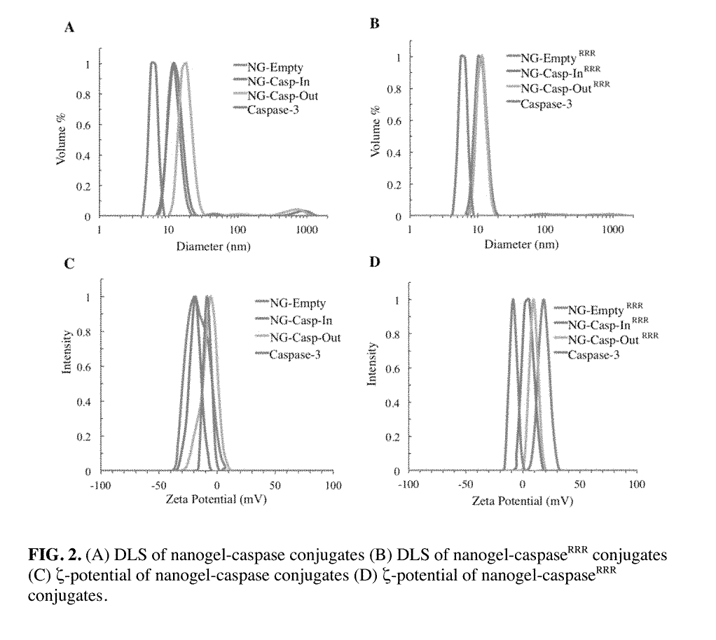

To further characterize these nanogel-protein conjugates, their size was evaluated by dynamic light scattering (DLS) and found that the hydrodynamic diameter of free caspase-3, NG-Empty and NG-Empty^{RRR} were 6, 12, and 10 nm respectively (FIG. 2). After the conjugation of caspase to the nanogels, there was an increase in the size of the conjugates, indicative of protein conjugation (12 nm-18 nm, FIGS. 2A and B). At pH 7.4, caspase-3 is negatively charged (pI value of 6.09), whereas CRRR is highly positively charged due to the arginine residues. These differences led us to evaluate the change in the surface charge of these conjugates by zeta potential measurements. After conjugation of caspase-3, the surface charge of NG-Casp-In (−19 mV) did not change significantly from the value observed in the original NG-Empty (−17 mV, FIG. 2C).

In the case of NG-Casp-Out, the zeta potential value obtained shifted towards −7 mV, similar to the value observed for free caspase-3 (−8 mV). After surface decoration of nanogels with CRRR, it was expected that NG-Empty^{RRR} would show a positive zeta potential value since these nanogels do not have caspase-3 conjugated. As expected, the surface charge for NG-Empty^{RRR} was found to be +18 mV. However, with caspase-3 conjugation, the surface charge became less positive: +4 mV for NG-Casp-In^{RRR} and +9 mV for NG-Casp-Out^{RRR} (FIG. 2D).

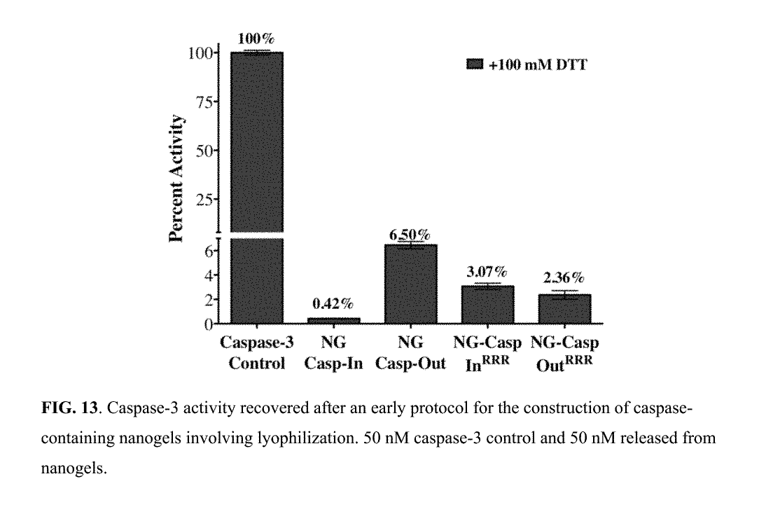

These results, along with the UV-vis absorption spectra recorded during the nanogel-caspase conjugates synthesis (FIGS. 12 and 13), suggest that unreacted PDS groups from the nanogels were sterically available to covalently react with the surface exposed cysteine residues from caspase-3. Furthermore, despite the presence of a large and bulky biomacromolecule on these polymeric nanogels, subsequent surface functionalization by CRRR addition can also be achieved, demonstrating the prevalent accessibility and reactivity of the PDS groups. The properties of these nanogel-caspase conjugates are summarized in Table 1.

TABLE 1
Summary of the Properties of Nanogels, Caspase-3
and Nanogel-Caspase Conjugates.

| | Crosslinking | | Zeta |
|---|---|---|---|
| System | Density (%) | Size (nm) | Potential (mV) |
| Caspase-3 | — | 6 | −8 |
| NG-Empty | 18 | 12 | −17 |
| NG-Casp-In | 18 | 12 | −19 |
| NG-Casp-Out | 18 | 18 | −7 |
| NG-Empty^{RRR} | 18 | 10 | 18 |
| NG-Casp-In^{RRR} | 18 | 12 | 4 |
| NG-Casp-Out^{RRR} | 18 | 12 | 9 |

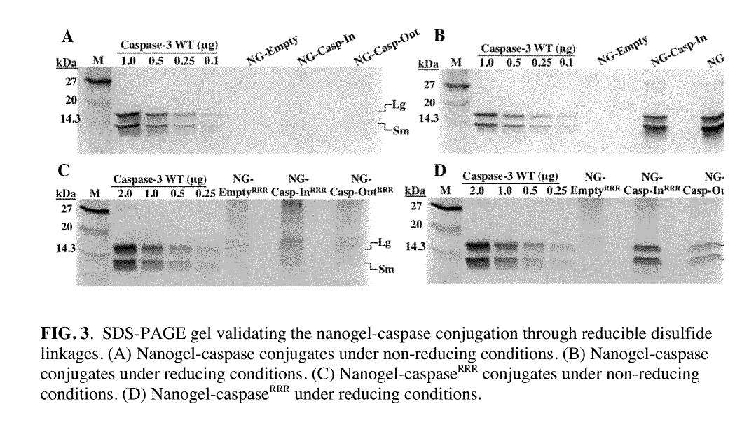

To further assess the protein conjugation to the nanogels, sodium dodecyl sulfate polyacrylamide gel electrophoresis (SDS-PAGE) was utilized to assess caspase dissociation from the nanogels in a reducing environment (FIG. 3). When digested with SDS and boiled, caspase-3 dissociates into the constituent large and small subunits (17 and 12 kDa, respectively), which migrate as two characteristic bands on SDS-PAGE. It was expected that caspase-3 covalently conjugated to the inside or outside of nanogels should not be observable by SDS-PAGE, because it will remain bound to and migrate (or fail to migrate) with the nanogel. It was expected to observe the two caspase-3 bands only after treatment of the nanogels with a reducing agent. As anticipated, not observed were the bands corresponding to caspase-3 in the NG-Casp-In nor NG-Casp-Out samples in the absence of reductant (FIG. 3A). This indicates that caspase-3 is covalently conjugated through disulfide bonds and not simply associated through physical adsorption.

Since disulfide bonds can be cleaved in the presence of high reducing agent concentrations, one should be able to “unlock” the protein from the assembly by exposing the conjugates to DTT. The appearance of the characteristic caspase-3 bands for the large and small subunits in both samples, NG-Casp-In and NG-Casp-Out, indicated that the protein was released after the reduction of disulfide bonds. This confirmed the conjugation of caspase-3 in both the “in” and “out” configurations (FIG. 3B) and demonstrated that these nanogels respond to a specific redox stimulus, namely DTT.

The concentration of caspase-3 released from each conjugate was estimated by comparing the intensity of the bands from nanogel-released caspase to known concentrations loaded into neighboring wells. Both nanogel-caspase conjugates (50 μg) released about 2μμg of caspase-3. Similar results were observed for the nanogels functionalized with the cell-penetrating peptide, RRR. Under non-reducing conditions, no caspase bands were observed for the NG-Casp-In^{RRR} and NG-Casp-Out^{RRR} samples (FIG. 3C). When the samples were exposed to reducing conditions, the appearance of the bands was observed that was indicative of free caspase-3 released from NG-Casp-In^{RRR} and NG-Casp-Out^{RRR} (FIG. 3D). The concentration of released caspase-3 from 50 μg NG-Casp-In^{RRR} and NG-Casp-Out^{RRR} was found to be 1 μg and 0.5 μg, respectively.

A capability demonstrated herein is that the proteins can be encapsulated within the interior of these nanogels or conjugated to the surface of the nanogels by simply altering the order of protein conjugation and crosslinking steps. Therefore, to determine if the caspase-3 was indeed predictably conjugated in the interior or on the surface of the nanogels an enzymatic degradation study was performed. The conjugates were exposed to acetonitrile (20% of the total volume) to denature the protein. A protein digest was then conducted by the addition of trypsin, a serine protease that hydrolyzes peptide bonds strictly after the basic residues arginine and lysine. (Polgar 2005 Cell. Mol. Life Sci. 62, 2161-2172.)

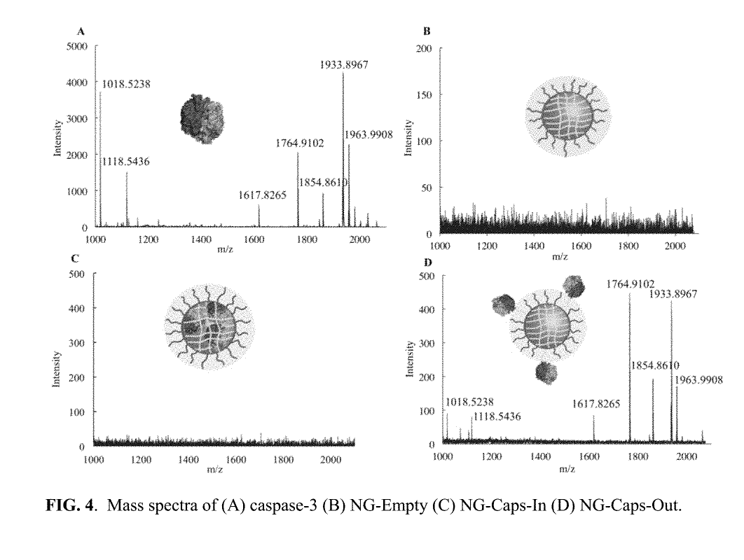

These fragments were then analyzed by mass spectrometry (MS). First, caspase-3 itself was subjected to the trypsin treatment conditions and the MS analysis displayed 7 major peaks with m/z values of 1018.5238, 1118.5436, 1617.8265, 1764.9102, 1854.8610, 1933.8967 and 1963.9908 (FIG. 4A). The caspase-3 fragments that each of these peaks represent are shown in Table 2.

TABLE 2
Summary of the Peptide Sequences of Caspase-3
Detected by Mass Spectrometry

| m/z (av) | Start | End | Sequence |
|---|---|---|---|
| 1018.5238 | 66 | 73 | (R)EEIVELMR(D) |
| 1118.5436 | 37 | 47 | (R)SGTDVDAANLR(E) |
| 1617.8265 | 61 | 73 | (K)NDLTREEIVELMR(D) |
| 1764.9102 | 197 | 210 | (K)QYADKLEFMHILTR(V) |
| 1854.861 | 30 | 47 | (K)STGMTSRSGTDVDAANLR(E) |
| 1933.8967 | 215 | 231 | (K)VATEFESFSDATFHAK(K) |
| 1963.9908 | 233 | 248 | (K)QIPCIVSMLTKELFY(-) |

Also analyzed were empty nanogels by the same method; as expected, no peaks were observed in the mass spectrum (FIG. 4B). In the case of the NG-Casp-In, if the caspase-3 was in fact encapsulated within the crosslinked core of the assembly, trypsin would not be able to reach the caspase-3 and therefore no signal would be observed. As predicted, none of the peaks observed previously for caspase-3 were present, suggesting that the protein is “wrapped-up” and protected from proteolytic digestion within the nanogels (FIG. 4C). On the other hand, in the case of NG-Casp-Out, peaks with m/z matching those observed for caspase-3 were detected, demonstrating that these caspase molecules are indeed on the outside of the nanogel assemblies and not protected from proteolysis like those in the NG-Casp-In state (FIG. 4D). These results demonstrate the versatility of these polymeric materials to covalently bind proteins and “cage” the cargo protein within the nanogel, protecting it from enzymatic degradation.

Encapsulated Caspases are Active upon Release

An attractive aspect of this system is the versatility of these polymeric nanogels to be decorated either at the surface with cargo proteins or to “wrap-up” intact active cargo proteins, and then release this cargo in response to a redox trigger. This capability is only useful if protein cargos retain enzymatic activity upon release from the nanogels. The enzymatic activities of caspase-3 conjugated to the nanogels or released after redox stimulus were assessed using a fluorogenic substrate cleavage assay.

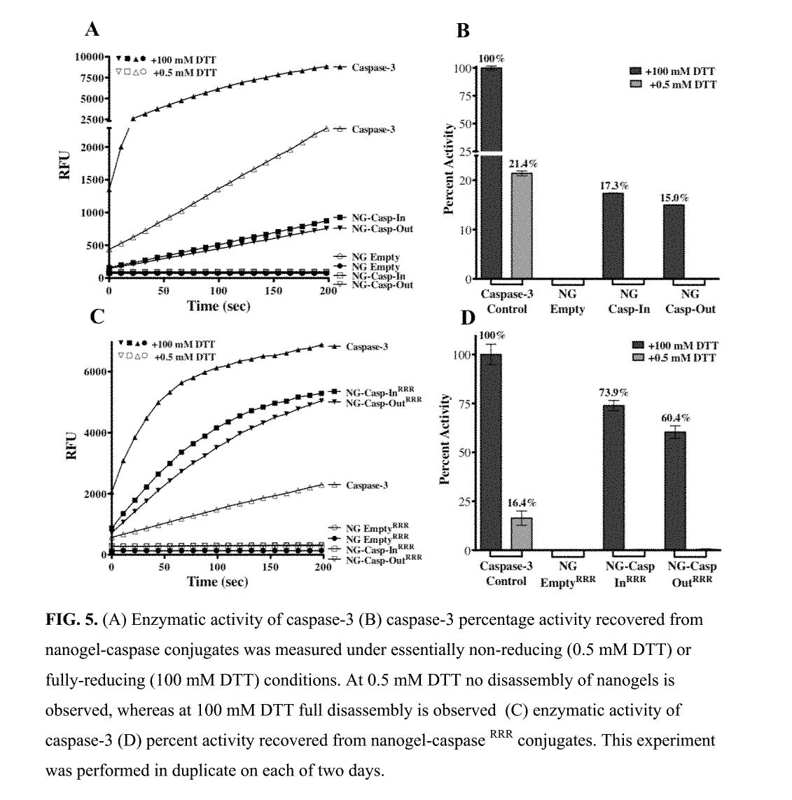

Based on quantification of caspase-release (FIG. 3), the activity from the precise quantity of nanogels encapsulating 50 nM caspase-3 was measured under non-reducing conditions (FIG. 5). Due to the fact that the catalytic cysteine of caspase-3 requires a reducing environment to be active, a small amount of DTT (0.5 mM) was added for the “non-reducing conditions.” This was enough reductant to assess activity of any unbound caspase, but not enough to promote disassembly of the nanogels.

Under these conditions, none of the conjugates exhibited caspase-3 activity, suggesting there is no unbound active caspase and the enzyme conjugated to the nanogel is effectively catalytically silenced. This silencing could be due to the structural constraints imposed by the nanogels and/or the lack of accessibility to the peptide substrate. The fact that no caspase-substrate cleavage was observed, even in the NG-Casp-Out nanogels, suggests that even non-encapsulated caspase have been catalytically silenced. Caspase-3 contains a free and highly reactive cysteine at its surface that is part of its catalytic diad (Cys-285). Due to its reactivity, it is likely that this cysteine could be one of the primary amino acids to be conjugated to the nanogel. Thus, in the case of surface incorporated caspase, the protein's observed activity could be silent due to covalent conjugation of the active site Cys-285.

The nanogel-caspase conjugates were then incubated in the presence of 100 mM DTT to release the caspase-3 cargo. Activity was observed after incubation with 100 mM DTT, confirming that the protein is active upon release from the nanogel (FIGS. 5A and C). Released caspase-3 activity reached 17% (FIG. 5B) upon release from NG-Casp-In and NG-Casp-Out and 74% for NG-Casp-In^{RRR} and NG-Casp-Out^{RRR} (FIG. 5D). An earlier protocol for the construction of caspase-containing nanogels utilized lyophilization. Caspase-3 is an obligate heterotetramer, which does not refold spontaneously with high yields, so it is not surprising that dramatically lower yields of functional protein (0.24-3%) were released from lyophilized nanogels using the older protocol (FIG. 13).

Given the 15-74% recovery rates observed currently, it is safe to conclude that the conjugation process itself is mild and that the nanogels protect the caspase, helping it to avoid denaturation and thus retain enzymatic activity. It was surprising to note the higher fraction of recovered caspase-3 activity upon release from the triarginine-containing nanogels, NG-Casp-In^{RRR} and NG-Casp-Out^{RRR} (FIG. 5D) than from NG-Casp-In and NG-Casp-Out (FIG. 5B). This result was puzzling, since both these types of nanogels had very similar abilities to induce cellular apoptosis (vide infra).

A key difference between these nanogels is that the non-functionalized nanogels (NG-Casp-In, NG-Casp-Out) contain unreacted PDS moieties, while these functional groups have been consumed during conjugation of the RRR peptide in the functionalized nanogels (NG-Casp-In^{RRR} and NG-Casp-Out^{RRR}). It was hypothesized that during release from the non-functionalized nanogels that the added DTT is initially increasing the percent crosslinking (toward 100%) leaving only a remaining fraction of DTT to liberate the caspase from what is then a much more extensively crosslinked nanogel. To test this hypothesis, the non-functionalized nanogel-conjugates were prepared as before, with an 18% crosslinking density and then reacted away the remaining PDS groups using thiol-terminated PEG (MW 1 k) to generate NG-Casp-In^{PEG} and NG-Casp-Out^{PEG}.

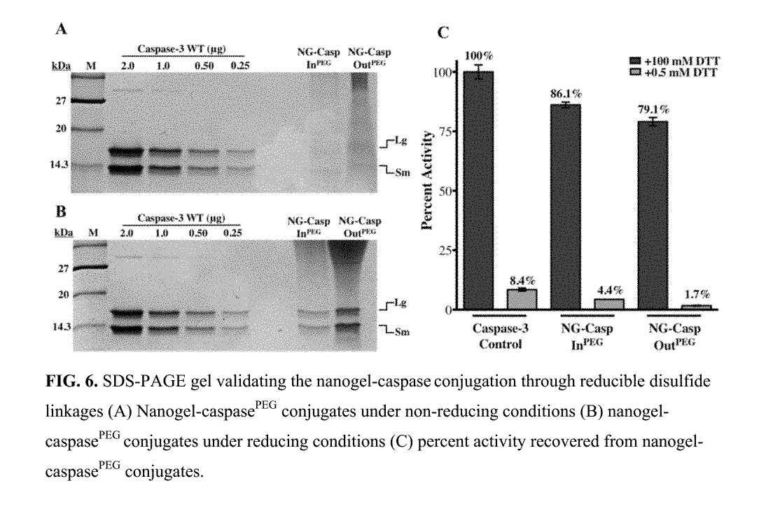

Next, the enzymatic activity of capase-3 was assessed after exposing the conjugates to 100 mM DTT. SDS-PAGE of the nanogel-caspase PEG-thiol conjugates validated the caspase conjugation (FIGS. 6A and B). Once the non-functionalized nanogel-conjugates were protected from additional crosslinking by the addition of PEG groups, it was possible to release and recover a high percentage of caspase activity (79% and 86%, FIG. 6C) comparable to those observed for the nanogel-caspase^{RRR} conjugates. The extent of recovered caspase-3 activity is quite remarkable considering the lability of the caspase heterotetramer and its dependence on proper formation of the active-site loop bundle for activity. This high yield indicates that these nanogels incorporate their cargo without damaging it, while at the same time remaining robust and responding specifically to a redox stimulus.

### Nanogels Promote Cell Internalization

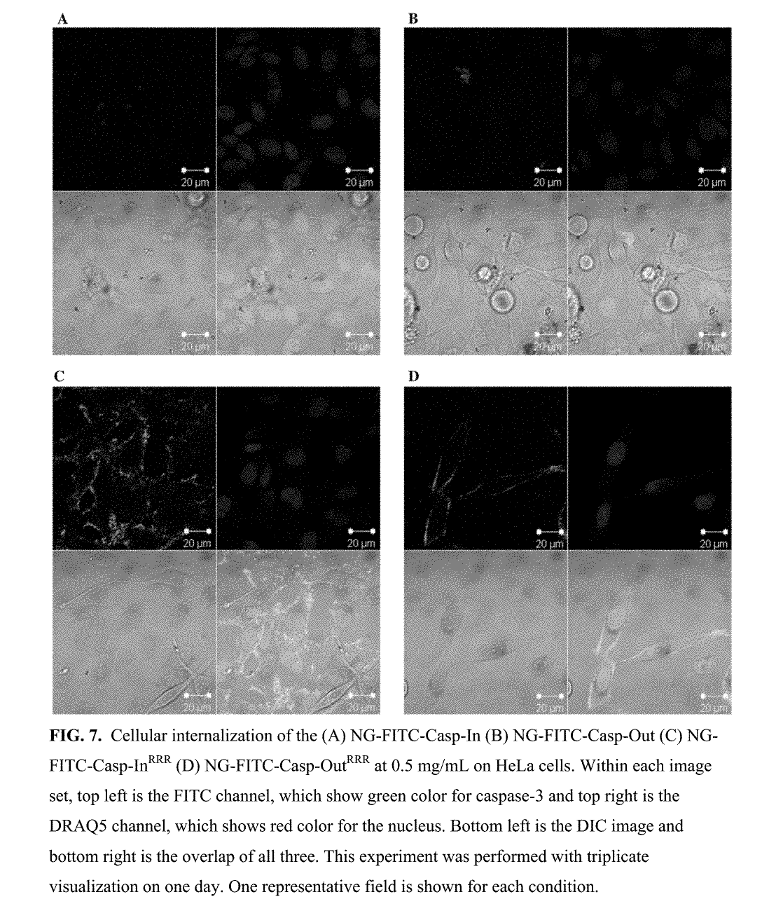

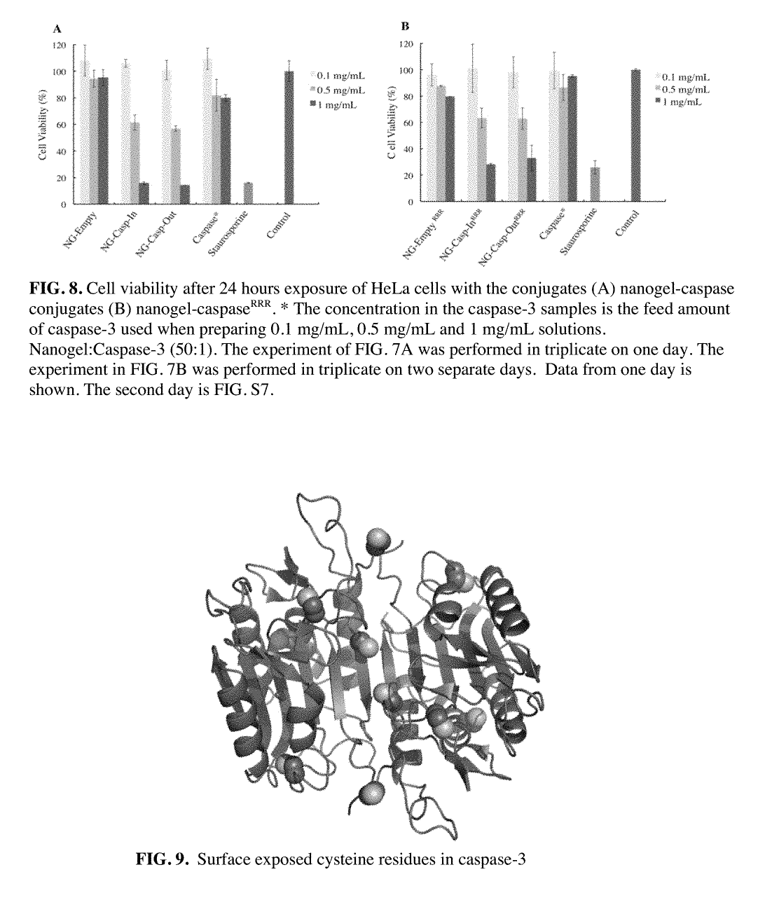

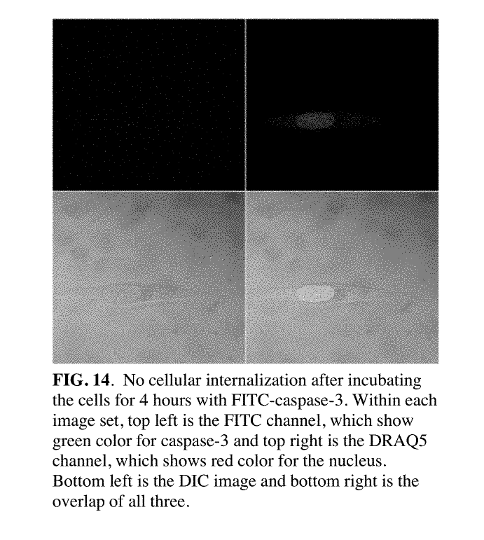

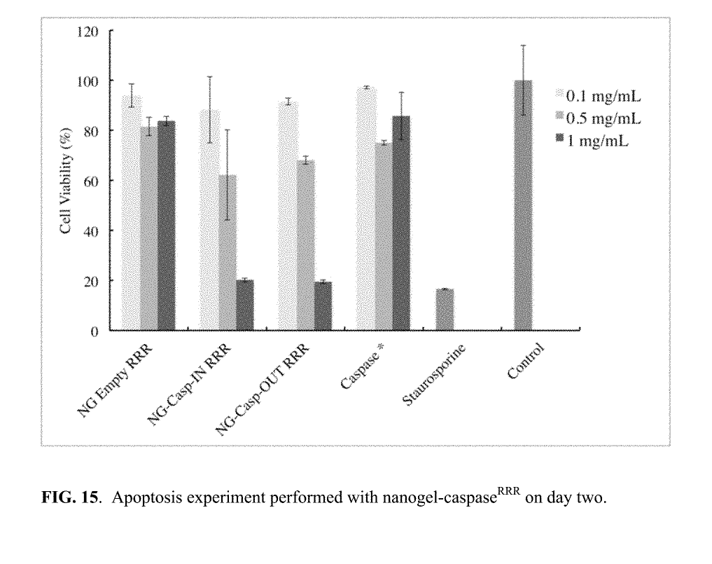

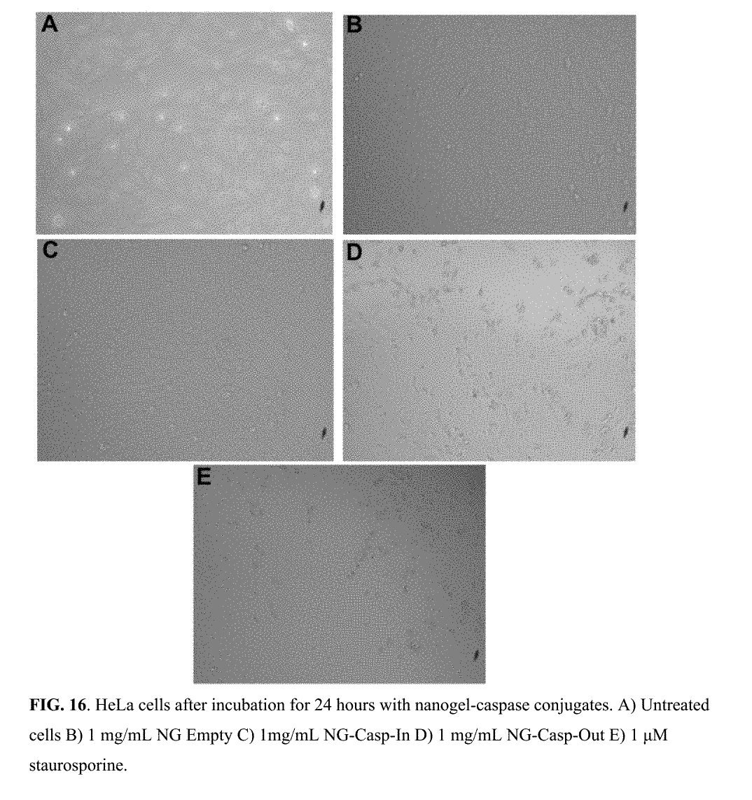

The objective for the use of these nanogel-protein conjugates is deliver enzymes in their inactive form and activate them using the innate intracellular environment in mammalian cells. In this case, such caspase delivery is expected to result in cell killing. To determine whether caspase-conjugated nanogels are capable of internalization in living cells, the cellular uptake of nanogels upon incubation of the conjugates with HeLa cells was monitored. Caspase-3 was labeled with fluorescein isothiocyanate (FITC) to enable intracellular visualization; the cell nucleus was stained with DRAQ5. The fluorescence distribution of FITC and DRAQ5 was observed by confocal fluorescence microscopy (FIGS. 7 and 14).

FITC-caspase-3 was observed for nanogels caspase on the surface or inside of nanogels (FIG. 14A, B), whereas no fluorescence was visible in cells treated with FITC-labeled caspase-3 (FIG. 14), suggesting that the protein is not capable of penetrating the cells by itself and requires nanogel conjugation for efficient internalization. Cellular uptake of nanogel conjugates is slow. This trend mirrors previous reports showing no significant internalization of related nanogels at doses of 0.1 mg/mL after 6 hours in HeLa cells. (Ryu, et al. 2012 Biomacromolecules 13, 1515-1522.)

To improve uptake, triarginine containing peptides were also incorporated into caspase-containing nanogels (NG-Casp-Out^{RRR} and NG-Casp-In^{RRR}, FIG. 1, Scheme 1) As predicted, nanogels functionalized with RRR displayed higher accumulation of caspase-3 both on the membrane and in the cell within this short time frame. This is likely due to the increased local concentration of the positively charged RRR and the negatively charged cellular membrane. Although there is no clear consensus as to the detailed mechanism by which the RRR-targeted assemblies are internalized, it is now widely accepted that arginine-rich peptides and their cargo are internalized via endocytosis. (Fuchs, et al. 2004 Biochemistry 43, 2438-2444; Nakase, et al. 2004 Mol. Ther. 10, 1011-1022; Nakase, et al. 2007 Biochemistry 46, 492-501.)

### Caspase-Nanogel Assemblies Induce Apoptotic Cell Death

Caspase-3 plays a critical role during the apoptotic process, so it was anticipated strong cell death-inducing potential of these nanogel-caspase conjugates, which release up to 75% of the caspase-3 cargo in an active form. The extent of cell death was measured in HeLa cells treated with increasing doses of the nanogel conjugates (FIG. 8). Staurosporine, a protein kinase inhibitor known to induce apoptosis was used as a positive control. After 24 hours, cell viability was measured using Alamar Blue assay. Apoptosis is characterized by the marked changes in cell morphology such as cell shrinkage and blebbing. (Zhang, et al. 2004 Mol. Cancer Ther. 3, 187-197; Porter, et al. 1999 Cell Death Differ. 2, 99-104.)

HeLa cells treated with nanogel caspase conjugates appear to be rounded and shrinking similar to as those undergoing apoptosis induced by staurosporine (FIG. 13), suggesting that killing was via an apoptotic route.

Bare nanogels would be relatively non-toxic and that those conjugated to caspase-3 should exhibit higher rates of cell death induction. NG-Empty exhibited low cellular toxicity at concentrations up to 1 mg/mL (FIG. 8A), whereas nanogel-caspase conjugates displayed a strong dose response for cell death. At a concentration of 1 mg/mL, the cell viability for both NG-Casp-In and NG-Casp-Out was reduced to nearly 20%. To confirm that the cell death observed in the nanogel-caspase conjugates was induced by the intracellular release of active caspase-3 aided by the nanogels and not by the action of caspase-3 in solution, cells were exposed to free caspase-3 utilizing the amount of protein fed during the synthesis of 0.1 mg/mL, 0.5 mg/mL and 1 mg/mL nanogels (50:1 weight ratio, nanogel:caspase-3).

Since caspase-3 alone is not expected to effectively penetrate the cell membrane, the protein itself should not induce cell death. As expected, the cell viability observed for caspase-3 was approximately 80% for a concentration up to 1 mg/mL, indicating that the vast majority of the cell death observed corresponded to apoptosis induced by the intracellular release of active caspase-3 from the polymeric nanogels. Similar results were observed for the case of nanogels decorated with RRR peptide (FIG. 8B). At a concentration of 1 mg/mL, the cell viability for NG-Empty^{RRR} was about 80% (FIG. 8B); this may be because positively charged RRR peptides directly penetrate the cell membrane, causing rupture or damage, thus introducing higher toxicity. (Prevette, et al. 2010 Mol. Pharmaceutics 7, 870-883.)

Similar to the nonfunctionalized nanogel caspase conjugates, the RRR nanogels induced cell death in a dose responsive manner. Cell viability for NG-Casp-In^{RRR} and NG-Casp-Out^{RRR} was about 30-35% at a concentration up to 1 mg/mL. Although nanogels lacking any targeting peptides (FIG. 7A, B) show much less cell internalization than the RRR nanogels, they are more capable of inducing cell death. At first this result was perplexing, with cell internalization studies seemingly uncorrelated with increases in cell death. However, upon further investigation it was observed that that the RRR-decorated nanogels appear to be accumulating on cell membranes, rather than being fully incorporated in the cytoplasm where caspases can be released and activated. (Jiao, et al. 2009 1 J. Biol. Chem. 284, 33957-33965.)

In particular, cationic cell penetrating peptides are known to enter cells via endocytosis. If indeed these nanogels are getting trapped in an endosome, they would be unable to release their cargo due to the oxidizing environment of this cell compartment. Even if the nanogel was disrupted, the oxidizing conditions and endosomal pH would render the caspase inactive. Caspase-3 is optimally active at pH 7.5 with a significant decrease in activity at pH values in the endosome (estimated to be from 5 to 6.5) dropping to just 10% remaining activity at pH 6.0. (Feener, et al. 1990 J. Biol. Chem. 265, 18780-18785; Austin, et al. 2005 Proc. Natl. Acad. Sci. 102, 17987-17992; Paroutis, et al. 2004 Physiology 19, 207-215; Geisow, et al. 1984 Exp. Cell. Res. 150, 36-46; Garcia-Calvo, et al. 1999 Cell Death Differ. 6, 362-369.)

Thus, although far fewer NG-Casp exist in cells and NG-Casp^{RRR} are more abundant overall, the NG-Casp have greater cell-killing potential, presumably due to their more favorable intracellular localization. This points to an important finding. Optimal delivery of caspase may require delivery materials capable of endosomal escape. The fact that the nanogels (without the cell penetrating peptides) exhibit excellent apoptotic efficiency suggests that these nanocarriers are already doing well.

### EXPERIMENTAL

Materials and Methods

Polyethylene glycol monomethyl ether methacrylate (PEGMA) (MW 475), 2,2′-dithiodipyridine, 2,2′-azobis(2-methylpropionitrile) (AIBN), 4-cyano-4-(phenylcarbonothioylthio) pentanoic acid (chain transfer agent), D,L-dithiothreitol (DTT), and other conventional reagents were obtained from commercial sources and were used without further purification, except for AIBN which was purified by recrystallization. Pyridyl disulfide ethyl methacrylate (PDSMA) was prepared using the previously reported procedure (Macromolecules, 2006, 39, 5595-5597).

¹H-NMR spectra were recorded on a 400 MHz Bruker NMR spectrometer using the residual proton resonance of the solvent as the internal standard. Chemical shifts are reported in parts per million (ppm).

Molecular weight of the polymer was estimated by gel permeation chromatography (GPC) in THF using the poly(methyl methacrylate) (PMMA) standard with a refractive index detector. Dynamic light scattering (DLS) measurements were performed using a Malvern Nanozetasizer.

UV-Visible absorption spectra were recorded on a Varian (Model EL 01125047) spectrophotometer.

Size exclusion chromatography was performed on Amersham Biosciences chromatography system equipped with a GE health care life sciences superdex 75 10/300 GL column.

Activity assay was performed utilizing Spectramax M5 spectrophotometer. MALDI-MS analyses were performed on a Bruker Autoflex III time-of-flight mass spectrometer. All mass spectra were acquired in the reflectron mode, and an average of 200 laser shots at an optimized power (60%) was used. Cell imaging was performed using Zeiss 510 META confocal microscope.

### Determination of the Number of Accessible and Reactive Cysteine (Thiols) on Caspase-3

A 1.5 mL stock of 10 μM caspase-3 was purified using a NAP25 (GE Healthcare) size exclusion column to fully exchange the buffer to 20 mM Tris pH 8.0 and eliminate all DTT. The caspase-3 (3.6 μM) was then incubated at room temperature with 50 μM of 5,5′-dithiobis-(2-nitrobenzoic acid) (DTNB) for 30 minutes. The absorbance of this reaction was then recorded at 412 nm. The Beer-Lambert law was then used on the corrected absorbance with a molar absorptivity for DTNB of 14,150 M⁻¹cm⁻¹ and a pathlength of 1 cm. This yielded 5 DTNB molecules per monomer of caspase, indicating that there are 5 accessible and reactive cysteine thiols per monomer of caspase-3.

Labeling of Caspase-3 with FITC

Fluorescein isothiocyanate isomer I (FITC) was dissolved in a 100 mM sodium bicarbonate solution pH 9.0 to a concentration of 1 mg/mL. Caspase-3 (2.65 mg) was diluted to 2 mL in 100 mM sodium bicarbonate buffer pH 9.0 with a total of 1.5 mg of FITC present. This reaction was protected from light and allowed to stir overnight at 4° C. The resulting FITC-labeled caspase-3 was dialyzed in 50 mM Tris pH 7.5, 50 mM NaCl, and 2 mM DTT to remove excess FITC. The caspase was then concentrated using a 3,000 Da spin filter and the concentration was measured by UV-vis absorption spectroscopy.

Synthesis of p(PEGMA-co-PDSMA) (P1)

A mixture of PDSMA (536.8 mg, 2.1 mmol), PEGMA (1 g, 2.1 mmol), 4-Cyano-4-(phenylcarbonothioylthio) pentanoic acid (21 mg, 0.0756 mmol) and AIBN (1.2 mg, 0.00756 mmol) were dissolved in 3 mL THF in a 10 mL Schlenk flask and degassed by performing three freeze-pump-thaw cycles with an argon inflow into the reaction. The reaction vessel was sealed and placed in a pre-heated oil bath at 70° C. for 12 hours. The product polymer P1 was then purified by precipitation in cold ether (20 mL) to yield the random copolymer. Yield: 90%. GPC (THF) Mₙ: 20 K. Ð: 1.5. ¹H-NMR (400 MHz, CDCl₃) δ: 8.45, 7.66, 7.09, 4.20-4.06, 3.80-3.42, 3.01, 2.10-1.65, 1.10-0.80. The molar ratios between the two blocks was determined by the integration of the methoxy proton in the polyethylene glycol unit and the aromatic proton in the pyridine and found to be 48%:52% (PEG:PDS).

### Synthesis of CRRR Peptide

This peptide was prepared following a previously reported procedure (Am. Chem. Soc., 2012, 134, 6964-6967) except utilizing a different resin. Rink Amide AM resin (200-400 mesh) was selected as the solid support to prepare the peptide using the solid phase synthesis. Peptide was used without further purification. ¹H NMR (400 MHz, D₂O) δ: 4.45-4.52, 4.30-4.41, 4.03-4.11, 3.15-3.26, 2.92-3.01, 1.73-1.96, 1.55-1.96. MS (FAB): exact mass calculated: 590.7. Found: 590.0

### Synthesis of the Nanogel-Caspase Conjugates

NG Empty: The polymer (3 mg) was dissolved in 1 mL 1× PBS buffer pH 7.4 and the solution was left stirring at 20° C. for 15 minutes. A measured amount of DTT was added to the aggregates and the solution was allowed to crosslink for 1 hour at 20° C. The resulting nanogels were dialysed at 20° C. using a 7,000 Da MWCO membrane.

NG-Casp-In: The polymer (3 mg) was dissolved in 1 mL 1× PBS buffer pH 7.4 and the solution was left stirring at 20° C. for 15 minutes. To this aggregate solution, 0.06 mg of caspase-3 was added and the mixture was left reacting for 1 hour at 20° C. A measured amount of DTT was added to the solution and was stirred for another hour at 20° C. to allow for crosslinking. The resulting nanogels were dialysed at 20° C. using a 7,000 Da MWCO membrane and unbound caspase-3 was removed by Amicon Ultra Centrifugal Filters MWCO 100,000.

NG-Casp-Out: The polymer (3 mg) was dissolved in 1 mL PBS buffer pH 7.4 and the solution was left stirring at 20° C. for 15 minutes. A measured amount of DTT was added to the aggregates and the solution was allowed to crosslink for 1 hour at 20° C. Then, 0.06 mg of caspase-3 was added and the mixture was left reacting for another hour at 20° C. The resulting nanogels were dialysed at 20° C. using a 7,000 Da MWCO membrane and unbound caspase-3 was removed by Amicon Ultra Centrifugal Filters MWCO 100,000.

NG Empty^{RRR}: To functionalize the surface of the nanogels, the same procedure described above for nanogels was followed. In addition, CRRR peptide (50% by weight compared to the polymer) was added and then stirred for another hour at 20° C. The resulting nanogels were dialysed at 20° C. using a 7,000 Da MWCO membrane.

NG-Casp-In^{RRR}: To functionalize the surface of the nanogels, the same procedure described above for NG-Casp-In was followed. In addition, an excess of the ligand (5 mg), CRRR, was added and then stirred for another hour at 20° C. The resulting nanogels were dialysed at 20° C. using a 7,000 Da MWCO membrane and unbound caspase-3 was removed by Amicon Ultra Centrifugal Filters MWCO 100,000 Da.

NG-Casp-Out^{RRR}: To functionalize the surface of the nanogels, the same procedure described above for NG-Casp-Out was followed. In addition, CRRR peptide (50% by weight compared to the polymer) was added and then stirred for another hour at 20° C. The resulting nanogels were dialysed at 20° C. using a 7,000 MWCO membrane and unbound caspase-3 was removed by Amicon Ultra Centrifugal Filters MWCO 100,000.

### Crosslinking Density, Caspase Conjugation and Peptide Functionalization

The crosslinking density was determined following the reported procedure (J. Am. Chem. Soc., 2012, 134, 6964-6967) by calculating the amount of the byproduct, pyridothione, and its known molar extinction coefficient (8.08×10³ M⁻¹ cm⁻¹ at 343 nm) (Bioconjugate Chem. 2006, 17, 1376-1384). The functionalization of the nanogels with CRRR peptide was evaluated by the further formation of pyridothione (increase in the absorption spectra at 343 nm).

### EXAMPLE 1

### Calculation of Crosslinking Density in NG-FITC-Casp-In

PEG:PDS=48:52

Molecular weight of the polymer repeat unit: 360.6 g/mol

From the nanogel synthesis reaction mixture, a solution of 0.1 mg/mL=0.05 mg/0.5mL was prepared (total volume of 0.5 mL).

1) Calculate mol of PDS in the solution:

[0.05 mg/(360.6 g/mol)]*(0.52)=7.2×10⁻⁸ mol PDS

2) Calculate mol of pyridothione:
By Beer-Lambert law, A=ϵbc, and the absorbance at 343 nm of the solution: 0.8274
Therefore, c=0.8274/[(8.08×10³ M⁻¹ cm⁻¹) (1 cm)]=1.02×10⁻⁴ M
Since a total volume of 0.5 mL was used, the final mol of pyridothione is=5.1×10⁻⁸ mol

3) It is 70.8 mol % of total PDS unit (7.2×10⁻⁸ mol). It was assumed that two pyridothione are from one disulfide formation and PDS unit is 52 mol % of total polymer:
(70.8% /2)*0.52=18.5% crosslinking density.

### EXAMPLE 2

### Calculation of Crosslinking Density in NG-FITC-Casp-In'

From the nanogel synthesis reaction mixture, a solution of 0.05 mg/mL was prepared (total volume of 1 mL).

1) Calculate mol of PDS in the solution:

[0.05 mg/(360.6 g/mol)]*(0.52)=7.2×10⁻⁸ mol PDS

2) Calculate mol of pyridothione:
By Beer-Lambert law, A=ϵbc, and the absorbance at 343 nm of the solution: 4.01×10⁻¹
Therefore, c=4.01×10⁻¹/[(8.08×10³ M⁻¹ cm⁻¹) (1 cm)]=4.96×10⁻⁵M
Since a total volume of 1 mL was used, the final mol of pyridothione is=4.96 x 10⁻⁸ mol

3) It is 68.9 mol% of total PDS unit (7.2×10⁻⁸ mol). It is assumed that two pyridothione are from one disulfide formation and PDS unit is 52 mol % of total polymer:
(68.9%/2)*0.52=17.9% crosslinking density.

### Sodium Dodecyl Sulfate Polyacrylamide Gel Electrophoresis (SDS-PAGE) Studies

Samples of NG-Empty (50 μg), NG-Casp-In (50 μg), NG-Casp-Out (50 μg), NG-Empty^{RRR} (50 μg), NG-Casp-In^{RRR} (50 μg) and NG-Casp-Out^{RRR} (50 μg) were run on a 16% acrylamide gel under non-reducing conditions (no DTT present). Identical samples were then treated with 100 mM DTT and allowed to sit at 25° C. for one hour before SDS-PAGE. Using a standard curve of known caspase concentrations the caspase released from the nanogels was then quantified using the Bio-Rad Image Lab™ software. This yielded the precise amount of caspase released per 50 μg of nanogel.

### Enzymatic Degradation Experiment

Sample solutions of nanogels and nanogel-caspase conjugates (1 mg/mL) with a final volume of 0.050 mL were prepared. The concentration of caspase-3 in the conjugate solutions was estimated to be 14 μM based on the feed amount during its synthesis (caspase-3 molecular weight=28,500). The protein was denatured by adding acetonitrile (20% of the total volume) and the solutions were left at room temperature for 10 minutes. The digestion of caspase-3 was performed by adding trypsin from porcine pancreas, 4:1 ratio (caspase-3:trypsin), and incubated for 20 hours at room temperature. Nanogels solution was exposed to the same amounts and conditions as those for nanogel-caspase conjugates.

The matrix solution was prepared by mixing 22.5 mg of α-cyano-hydroxycinnamic acid in 350 μL tetrahydrofuran, 150 μL water and 6 μL of trifluoroacetic acid. 10 μL of each of the sample solutions and 10 μL of the matrix solution were mixed and spotted on a MALDI target.

### Activity Assay

For measurement of caspase-3 activity, milligrams of nanogel used was varied in order to release 50 nM caspase-3 in each experiment. These nanogel-caspase conjugates were incubated in 100 mM DTT for 1 hour in order to fully release the cargo caspase-3. Identical samples were subject to 0.5 mM DTT treatment in an identical fashion in order to assay for any free, or unbound, caspase-3. The caspase-3 activity was then assayed over a 7 minute time course in caspase-3 activity assay buffer containing 20 mM HEPES pH 7.5, 150 mM NaCl, 5 mM CaCl₂, and 10% PEG 400. In this experiment, caspase-3 hydrolyzes the peptide substrate, N-acetyl-Asp-Glu-Val-Asp-7-amino-4-methylcoumarin, resulting in the release of the 7-amino-4-methylcoumarin (AMC) moiety as a fluorophore that can be quantified over time. The fluorogenic substrate N-acetyl-Asp-Glu-Val-Asp-AMC, Enzo Lifesciences (Ac-DEVD-AMC), Ex 365/Em495, was added to a final concentration of 100 μM to initiate the reaction. These 100 μL assays were performed in duplicate in a 96-well microplate at 37° C. using a spectramax M5 spectrophotometer.

### Cell Internalization Studies

Cell internalization studies were performed using Zeiss 510 META confocal laser scanning microscopy. HeLa cells were cultured in T75 cell culture flask containing Dulbecco's

Modified Eagle Medium/Nutrient Mixture F-12 (DMEM/F12) with 10% fetal bovine serum (FBS) supplement. The cells were seeded at 100,000 cells/mL in cover slip-bottomed Petri dishes and allowed to grow for 1 day at 37° C. in a 5% CO₂ incubator. The cells in 1 mL of culture medium were treated with nanogels and nanogel-FITC-caspase conjugates (0.5 mg/mL) and were incubated for 4 hours at 37° C. before monitoring the cells by confocal microscopy. The nucleus of the cells was stained by the addition of DRAQ5 (5 μM). All images were taken using 63x oil immersion objectives (excitation at 488 nm for FITC and 543 nm for DRAQ-5).

### Apoptosis Studies

HeLa cells were cultured in T75 cell culture flasks using DMEM/F12 with 10% FBS supplement. The cells were seeded at 10,000 cells/well/200 μL in a 96 well tissue culture plate and allowed to grow for 24 hours under incubation at 37° C. in 5% CO₂. The cells were then treated with different concentrations of nanogel-caspase conjugates and were incubated for another 24 hours. Cell viability was measured using Alamar Blue assay with each data point measured in triplicate. Fluorescence measurements were made using the plate SpectraMax M5 by setting the excitation wavelength at 560 nm and monitoring emission at 590 nm on a black 96 well flat bottom plate.

In this specification and the appended claims, the singular forms “a,” “an,” and “the” include plural reference, unless the context clearly dictates otherwise. Unless defined otherwise, all technical and scientific terms used herein have the same meaning as commonly understood too one of ordinary skill in the art. Unless defined otherwise, all technical and scientific terms used herein have the same meaning as commonly understood by one of ordinary skill in the art. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A crosslinked polymeric nanogel-protein conjugate adapted to stably transporting a protein across a cell membrane and then intracellularly releasing the protein with intact biological activity.

2. The crosslinked polymeric nanogel-protein conjugate of claim 1, wherein the protein is securely encapsulated inside an aggregate formed by the self-crosslinked polymeric nanogel.

3. The crosslinked polymeric nanogel-protein conjugate of claim 1, wherein the protein is securely conjugated on a surface of the nanoaggregate formed by the self-crosslinked polymeric nanogel.

4. The crosslinked polymeric nanogel-protein conjugate of claim 2, wherein the protein is encapsulated inside the nanoaggregate by one or more disulfide linkages.

5. The crosslinked polymeric nanogel-protein conjugate of claim 3, wherein the protein is conjugated to the surface of the nanoaggregate by one or more disulfide linkages.

6. The crosslinked polymeric nanogel-protein conjugate of claim 1, wherein the protein is a cytosolically active protein.

7. The crosslinked polymeric nanogel-protein conjugate of claim 1, wherein the protein is caspase.

8. The crosslinked polymeric nanogel-protein conjugate of claim 1, wherein the conjugate is further functionalized with a targeting ligand.

9. The crosslinked polymeric nanogel-protein conjugate of claim 8, wherein the targeting ligand is an antibody protein, a peptide, or a small molecule.

10. The crosslinked polymeric nanogel-protein conjugate of claim 8, wherein the targeting ligand is a peptide.

11. The crosslinked polymeric nanogel-protein conjugate of claim 8, wherein the conjugate is functionalized with a cell penetrating peptide.

12. The crosslinked polymeric nanogel-protein conjugate of claim 1, wherein the polymeric nanogel comprises a random copolymer.

13. The crosslinked polymeric nanogel-protein conjugate of claim 1, wherein the polymeric nanogel is formed by reversible addition-fragmentation chain transfer polymerization of oligo(ethylene glycol) methacrylate and pyridyldisulfide methacrylate.

14. A method for controlled delivery of a protein to a target biological site inside a cell, comprising:
providing a crosslinked polymeric nanogel-protein conjugate;
delivering the crosslinked polymeric nanogel-protein conjugate intracellularly to the target biological site; and
causing a dissociation of the protein from the polymeric nanogel-protein conjugate resulting in intracellular release of the protein at the target biological site.

15. The method of claim 14, wherein the protein is a cytosolically active protein.

16. The method of claim 14, wherein the protein is caspase.

17. The method of claim 14, wherein the protein is securely encapsulated inside a nanoaggregate formed by the self-crosslinked polymeric nanogel.

18. The method of claim 14, wherein the protein is securely conjugated on a surface of a nanoaggregate formed by the self-crosslinked polymeric nanogel.

19. The method of claim 17, wherein the protein is encapsulated inside the nanoaggregate by one or more disulfide linkages.

20. The method of claim 18, wherein the protein is conjugated to the surface of the nanoaggregate by one or more disulfide linkages.

21-27. (canceled)
